(19) United States
(12) Patent Application Publication
(10) Pub. No.: US 2021/0074407 A1
(43) Pub. Date: Mar. 11, 2021

(54) SYSTEM TO FACILITATE INTEGRATION OF DATA WITH MEDICAL IMAGES

(71) Applicants: Siemens Healthcare GmbH, Erlangen (DE); New York University School of Medicine, New York, NY (US)

(72) Inventors: Bari Dane, New York, NY (US); Thomas O'Donnell, New York, NY (US)

(21) Appl. No.: 16/561,380

(22) Filed: Sep. 5, 2019

(51) Int. Cl. G16H 30/20; G06T 11/60; G06T 7/70

(52) U.S. Cl. CPC G16H 30/20; G06T 11/60; G06T 2207/30204; G06T 2210/41; G06T 7/70

(57) ABSTRACT

A system and a method include acquisition of medical image data of an interval volume of a body, acquisition of camera image data of the body, the camera image data comprising an indication of diagnosis-related information, integration of the medical image data and the camera image data to generate integrated medical image data, and display of the integrated medical image data.

### BACKGROUND

A radiologist uses medical images to diagnose and treat injuries and diseases. Medical images may be accompanied by notes of a referring physician, and the radiologist may use such notes to assist in the diagnosis and treatment. Quite often the notes are insufficient, unclear, incomplete, or, at worst, incorrect.

For example, the notes may indicate that a patient experiences pain in the left lower quadrant of the abdomen. Since the left lower quadrant can be quite large, such an indication may not be helpful to the radiologist. In another example, a patient may demonstrate a body movement which causes pain, and the referring physician will unsuccessfully attempt to draft a note which accurately captures the entire body movement. Even if sufficiently-accurate and relevant notes are provided, the reading of notes and contemporaneous viewing of corresponding images is an inefficient approach for absorbing the totality of information therein.

Systems are desired to improve the accuracy and/or relevance of the information communicated to the radiologist. Systems are also desired which efficiently integrate this information with medical images to facilitate the radiologist's understanding thereof.

### BRIEF DESCRIPTION OF THE DRAWINGS

FIG. 1 is a block diagram of a system to integrate external camera data with medical image data according to some embodiments;

FIG. 2A is a block diagram of a system to convert external camera data to a medical image format and merge the converted data with medical image data according to some embodiments;

FIG. 2B is a block diagram of a system to identify markers in external camera data and modify medical image data based on the markers according to some embodiments;

FIG. 3 is a block diagram illustrating a medical imaging system according to some embodiments;

FIG. 4 is a flow diagram of a process to convert external camera data to a medical image format and merge the converted data with medical image data according to some embodiments;

FIG. 5A illustrates an external camera image according to some embodiments;

FIG. 5B illustrates multiple external camera images according to some embodiments;

FIG. 6 is a view of displayed integrated medical image data according to some embodiments;

FIG. 7 is a flow diagram of a process to identify markers in external camera data and modify medical image data based on the markers according to some embodiments;

FIG. 8A illustrates an external camera image including an external marker according to some embodiments;

FIG. 8B illustrates multiple external camera images including an animated external marker according to some embodiments;

FIG. 9 is a view of displayed integrated medical image data according to some embodiments; and

FIG. 10 is a view of displayed integrated medical image data according to some embodiments.

### DETAILED DESCRIPTION

The following description is provided to enable any person in the art to make and use the described embodiments and sets forth the best mode contemplated for carrying out the described embodiments. Various modifications, however, will remain apparent to those in the art.

Some embodiments provide integration of camera data with medical image data. In one example, an external camera acquires one or more images of an external view of a body during and/or temporally proximate to acquisition of medical image data. The medical image data may comprise data representing an internal volume of the body and may be acquired using any imaging modality, including but not limited to X-ray, computed tomography (CT), magnetic resonance imaging (MRI), nuclear medicine, positron emission tomography (PET), single-photon emission computed tomography (SPECT) and ultrasound.

The camera data may capture patient gestures (e.g., pointing to an area of the body, indicating a path across the body, or other cue), markers placed on the patient (e.g., a circle around a rash or area of pain), or any other item or movement in the field of view of the camera. These gestures, markers, items and movements may be intended to convey information to a radiologist which may facilitate diagnosis and/or treatment based on the medical images. The camera data is integrated with the medical image data, and the integrated data is provided to a radiologist for viewing. The integrated data facilitates the delivery of the information represented by the camera data to the radiologist and may be viewed and absorbed efficiently thereby.

In some embodiments, the camera data is converted to a format suitable for use by an image viewing system of a radiologist (e.g., Digital Imaging and Communications in Medicine (DICOM) format). This format may be identical to the format of the medical image data with which the converted camera data will be integrated. Accordingly, the radiologist may view the camera data as a, e.g., DICOM series, on a viewing terminal in the same manner as viewing other DICOM series (e.g., axial, coronal, sagittal planes) of the medical image data.

In other embodiments, one or more markers (e.g., marks drawn on the patient) are identified from the camera data. The acquired medical image data is then modified based on the markers. For example, reconstruction of CT projections may take into account the location of the markers and include voxels representing the markers in the reconstructed CT volume. The reconstructed CT volume may then be viewed (e.g., as a DICOM series) as is known, in which the voxels representing the markers will be visible to the radiologist.

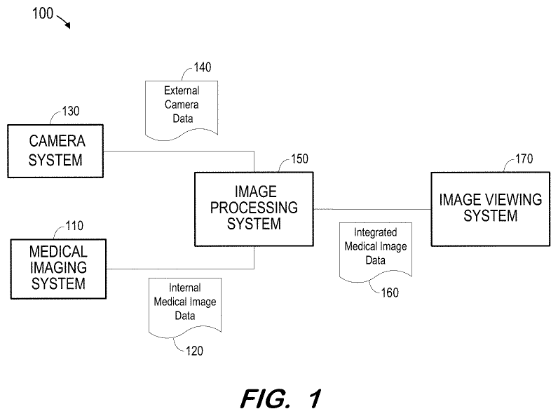

FIG. 1 is a block diagram illustrating system 100 according to some embodiments. Medical imaging system 110 may comprise any imaging system capable of acquiring one or more images of an internal patient volume that is or becomes known. Medical imaging system 110 may comprise one or more X-ray, CT, MRI, nuclear medicine, PET, SPECT and ultrasound systems in some embodiments.

The images acquired by medical imaging system 100 are represented in FIG. 1 as internal medical images data 120. Internal medical image data 120 may conform to any suitable data format and may undergo or have undergone any suitable processing, such as but not limited to motion correction, noise reduction, compression and reconstruction.

Camera system 130 may comprise any system to acquire an image of a patient during or temporally proximate to imaging by medical imaging system that is or becomes known. Camera system 130 may comprise a still camera, a video camera, a depth camera, an infrared camera, etc. External camera data 140 may confirm to any suitable data format generated by camera system 130. Camera system 130 may be mounted in a known orientation with respect to medical imaging system 110 so that external camera data 140 and internal medical image data 120 may be registered with one another if desired.

As described above, external camera data 140 may capture visual cues before, during, and/or after imaging by medical imaging system 110. Such visual cues may be intended to convey information to a radiologist who views camera data 140 in conjunction with internal medical image data 120. The information thusly-conveyed may facilitate diagnosis and/or treatment.

Image processing system 150 receives internal medical image data 120 and external camera data 140. Image processing system 150 integrates the received data to generate integrated medical image data 160. Integrated medical image data 160 includes information captured by external camera data 140 and by internal medical image data 120.

Integrated medical image data 160 is provided to image viewing system 170. Image viewing system 170 may comprise a picture archiving and communication system (PACS) as is known in the art. A radiologist may then operate image viewing system 170 to view integrated medical image data 160. By viewing integrated medical image data 160, the radiologist is provided with the information captured by external camera data 140 and by internal medical image data 120.

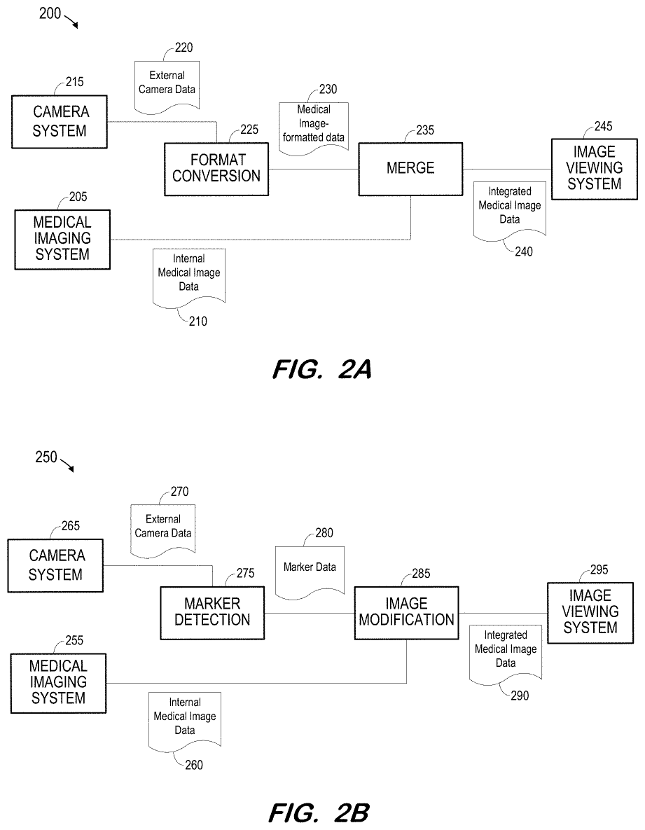

FIG. 2A is a block diagram illustrating system 200 according to some embodiments. System 200 may comprise an implementation of system 100 of FIG. 1, but embodiments are not limited thereto.

System 200 includes medical imaging system 205, which may operate and be implemented to generate internal medical image data 210 as described above with respect to system 110 and internal medical image data 120. Camera system 215 may comprise any system to acquire an image of a patient as described with respect to camera system 130. External camera data 220 may confirm to any suitable data format generated by camera system 215.

External camera data 220 may capture patient gestures (e.g., pointing to an area of the body, indicating a path across the body, or other cue), markers placed on the patient (e.g., a circle around a rash or area of pain), or any other item or movement in the field of view of camera system 215. These gestures, markers, items and movements may be intended to convey information to a radiologist which may facilitate diagnosis and/or treatment based on the medical images. Such information may include locations and/or directions of pain or other sensations, indications of physical limitations such as a region in which the patient's field of view is limited, etc.

External camera data 220 is provided to format conversion component 225. Format conversion component 225 converts external camera data 220 to medical image-formatted data 230. Medical image-formatted data 230 conforms to a format used in medical imaging and/or medical image viewing. The medical imaging format may be the same format to which internal medical image data 210 conforms. In some embodiments, internal medical image data 210 and medical image-formatted data 230 both conform to DICOM format. For example, medical image-formatted data 230 may comprise a DICOM series generated based on video image data (i.e., a sequence of still frames) of external camera data 220, and internal medical image data 210 may comprise one or more DICOM series output by medical imaging system 205.

Medical image-formatted data 230 and internal medical image data 210 are merged by merge component 235. In one example, merging may simply consist of adding the DICOM series of medical image-formatted data 230 to a file including the one or more DICOM series of internal medical image data 210. Merging generates integrated medical image data 240, which is provided to and viewed on image viewing system 245 as described above. Accordingly, format conversion component 225 and merge component 235 may comprise an implementation of image processing system 150 of FIG. 1. In some embodiments, a radiologist may then view the integrated medical image data 240 on image viewing system 245 as individual DICOM series (e.g., axial plane, coronal plane, sagittal plane, external camera video).

FIG. 2B is a block diagram illustrating system 250 according to some embodiments. System 250 may comprise an implementation of system 100 of FIG. 1, but embodiments are not limited thereto.

System 250 includes medical imaging system 255, which may also operate and be implemented to generate internal medical image data 260 as described above with respect to system 110 and internal medical image data 120. Similarly, camera system 265 may comprise any system to acquire an image of a patient as described with respect to camera system 130. External camera data 270 may confirm to any suitable data format generated by camera system 615.

External camera data 220 may capture images of markers placed on the patient (e.g., a circle around a rash or area of pain) in some embodiments. These markers may be intended to convey information to a radiologist which may facilitate diagnosis and/or treatment based on the medical images. In this regard, marker detection component 275 receives external camera data 270, detects one or more markers represented by data 270, and generates marker data 280 indicative of the detected marker(s). Marker data 280 may indicate a location of a marker, a size of a marker, a color of a marker, a code or other descriptive information (e.g., pain here) corresponding to a marker, and/or a direction in which a marker was drawn, for example.

Image modification component 285 receives marker data 280 and internal medical image data 260. In some embodiments, image modification component 285 modifies internal medical image data 260 based on marker data 280 to generate integrated medical image data 290. Image modification component 285 and marker detection component 275 may therefore comprise an implementation of image processing system 150 of FIG. 1.

Modification of internal medical image data 260 may comprise reconstruction of CT projections of internal medical image data 260 to account for the location of markers identified in marker data 280, and/or to include voxels representing the markers in the reconstructed CT volume. The reconstructed CT volume may then be presented by image viewing system 295 (e.g., as a DICOM series), in which the voxels representing the markers will be visible to the radiologist.

Each component of system 100, system 200 and system 250 may be implemented using any combination of hardware and software. For example, according to some embodiments, a computing system executes software code to provide functions attributed herein to image processing system 150. A single computing system may implement two or more of the illustrated components.

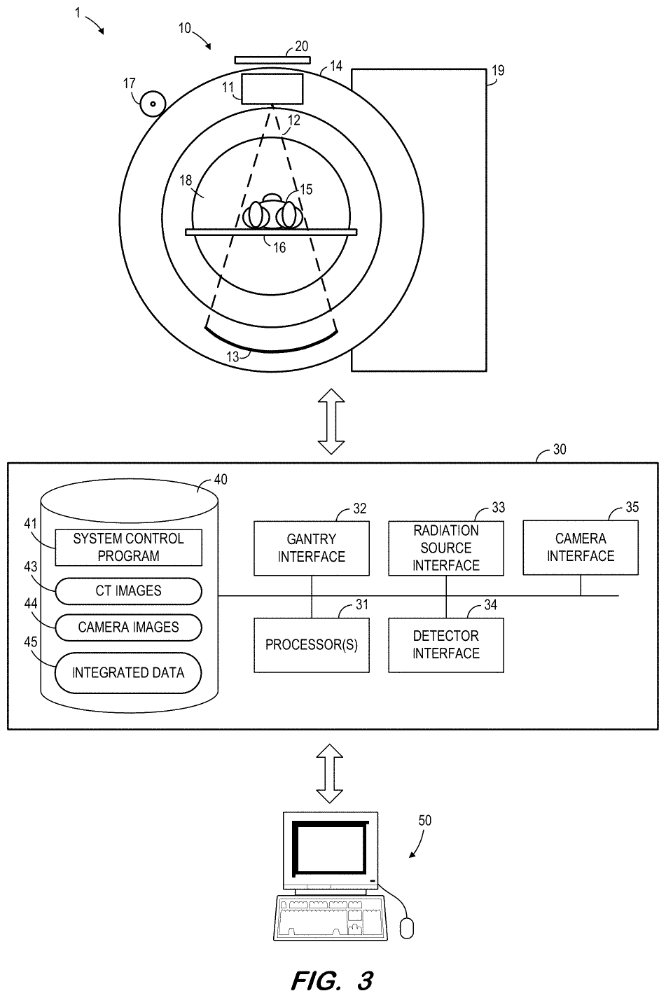

FIG. 3 illustrates system 1 according to some embodiments. System 1 may be operated to acquire internal medical image and external image data, and to generate integrated data based thereon according to some embodiments. Embodiments are not limited to system 1 to perform either function.

System 1 includes X-ray imaging system 10, camera 20, control and processing system 30, and operator terminal 50. Generally, and according to some embodiments, X-ray imaging system 10 acquires two-dimensional X-ray images (i.e., internal medical image data) of a volume of patient 15 and camera 20 acquires surface images of patient 15. Control and processing system 30 controls X-ray imaging system 10 and camera 20 to acquire image data, and receives the acquired image data therefrom. Control and processing system 30 may operate to integrate the image data. Such processing may be based on user input received by terminal 50 and provided to control and processing system 30 by terminal 50.

Imaging system 10 according to the example embodiment of FIG. 3 comprises a CT scanner including X-ray source 11 for emitting X-ray beam 12 toward opposing radiation detector 13. Embodiments are not limited to CT data or to CT scanners. X-ray source 11 and radiation detector 13 are mounted on gantry 14 such that they may be rotated about a center of rotation of gantry 14 while maintaining the same physical relationship therebetween.

Radiation source 11 may comprise any suitable radiation source. In some embodiments, radiation source 11 emits electron, photon or other type of radiation having energies ranging from 50 to 150 keV. Radiation detector 13 may comprise any system to acquire an image based on received X-ray radiation. In some embodiments, radiation detector 13 is a flat-panel imaging device using a scintillator layer and solid-state amorphous silicon photodiodes deployed in a two-dimensional array. The scintillator layer receives photons and generates light in proportion to the intensity of the received photons. The array of photodiodes receives the light and records the intensity of received light as stored electrical charge.

In other embodiments, radiation detector 13 converts received photons to electrical charge without requiring a scintillator layer. The photons are absorbed directly by an array of amorphous selenium photoconductors. The photoconductors convert the photons directly to stored electrical charge. Radiation detector 13 may comprise a CCD or tube-based camera, including a light-proof housing within which are disposed a scintillator, a mirror, and a camera.

The charge developed and stored by radiation detector 13 represents radiation intensities at each location of a radiation field produced by X-rays emitted from radiation source 11. The radiation intensity at a particular location of the radiation field represents the attenuative properties of mass (e.g., body tissues) lying along a divergent line between radiation source 11 and the particular location of the radiation field. The set of radiation intensities acquired by radiation detector 13 may therefore represent a two-dimensional projection image of this mass.

To generate X-ray images, patient 15 is positioned on bed 16 to place a portion of patient 15 between X-ray source 11 and radiation detector 13. Next, X-ray source 11 and radiation detector 13 are moved to various projection angles with respect to patient 15 by using rotation drive 17 to rotate gantry 14 around cavity 18 in which patient 15 is positioned. At each projection angle, X-ray source 11 is powered by high-voltage generator 19 to transmit X-ray radiation 12 toward detector 13. Detector 13 receives the radiation and produces a set of data (i.e., a raw X-ray image) for each projection angle.

Camera 20 may comprise any type of camera that is or becomes known. In some embodiments, camera 20 comprises a depth camera as is known in the art. A depth camera may comprise a structured light-based camera (e.g., Microsoft Kinect or ASUS Xtion), a stereo camera, or a time-of-flight camera (e.g., Creative TOF camera) according to some embodiments. Such a depth camera, mounted in a single stationary position, may acquire image data which consists of a two-dimensional image (e.g., a two-dimensional RGB image, in which each pixel is assigned a Red, a Green and a Blue value), and a depth image, in which the value of each pixel corresponds to a depth or distance of the pixel from the depth camera. This image data, consisting of a two-dimensional image and a depth image, is sometimes referred to as a two-dimensional depth image.

System 30 may comprise any general-purpose or dedicated computing system. Accordingly, system 30 includes one or more processors 31 configured to execute processor-executable program code to cause system 30 to operate as described herein, and storage device 40 for storing the program code. Storage device 40 may comprise one or more fixed disks, solid-state random access memory, and/or removable media (e.g., a thumb drive) mounted in a corresponding interface (e.g., a USB port).

Storage device 40 stores program code of system control program 41. One or more processors 31 may execute system control program 41 to move gantry 14, to move table 16, to cause radiation source 11 to emit radiation, to control detector 13 to acquire an image, to control camera 20 to acquire an image, and to perform any other function. In this regard, system 30 includes gantry interface 32, radiation source interface 33 and camera interface 35 for communication with corresponding units of system 10.

Two-dimensional X-ray data acquired from system 10 may be stored in data storage device 40 as CT image data 43, in DICOM or another data format. Each set of image data 43 may be further associated with details of its acquisition, including but not limited to time of acquisition, imaging plane position and angle, imaging position, radiation source-to-detector distance, patient anatomy imaged, patient position, contrast medium bolus injection profile, X-ray tube voltage, image resolution and radiation dosage. CT image data 43 may also include three-dimensional CT image data reconstructed from corresponding two-dimensional CT images as is known in the art.

Device 40 also stores two-dimensional camera image data 44 acquired by camera 20. In some embodiments, certain camera image data 44 may be associated with a set of CT image data 43, in that the associated image data was acquired at similar times while patient 15 was lying in substantially the same position. One or more processors 31 may execute system control program 41 to generate integrated data 45 based on CT image data 43 and corresponding camera image data 44 as described herein.

Terminal 50 may comprise a display device and an input device coupled to system 30. Terminal 50 may display any of CT images 43, camera images 44, and integrated data 44, and may receive user input for controlling display of the images, operation of imaging system 10, and/or the processing described herein. In some embodiments, terminal 50 is a separate computing device such as, but not limited to, a desktop computer, a laptop computer, a tablet computer, and a smartphone.

Each of system 10, scanner 20, system 30 and terminal 50 may include other elements which are necessary for the operation thereof, as well as additional elements for providing functions other than those described herein.

According to the illustrated embodiment, system 30 controls the elements of system 10. System 30 also processes image data received from system 10. Moreover, system 30 receives input from terminal 50 and provides images to terminal 50. Embodiments are not limited to a single system performing each of these functions. For example, system 10 may be controlled by a dedicated control system, with the acquired image data being provided to a separate image processing system over a computer network or via a physical storage medium (e.g., a Flash drive).

Embodiments are not limited to a CT scanner and a depth camera as described above with respect to FIG. 1.

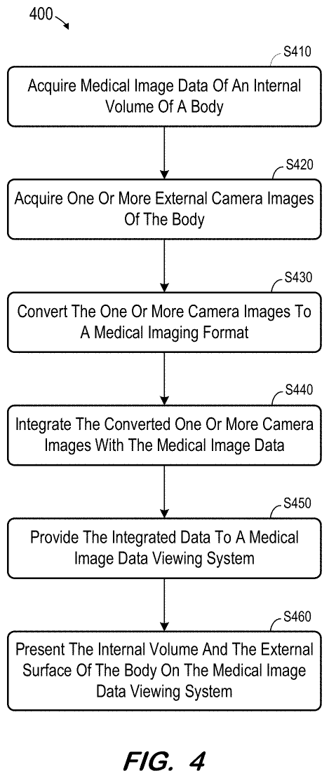

FIG. 4 is a flow diagram of process 400 according to some embodiments. Process 400 and the other processes described herein may be performed using any suitable combination of hardware, software or manual means. Software embodying these processes may be stored by any non-transitory tangible medium, including a fixed disk, a DVD, a Flash drive, or a magnetic tape. Embodiments are not limited to the examples described below.

Process 400 of FIG. 4 may be implemented by system 200 of FIG. 2A, but embodiments are not limited thereto. Initially, at S410, medical image data of an internal volume of a body is acquired. In one example as described above, medical imaging system 205 is operated at S410 to generate two- and/or three-dimensional internal medical image data 210 of a patient volume as is known in the art. Substantially contemporaneously, camera system 215 is operated to acquire one or more camera images of an external surface of the patient. The acquired medical image data and the acquired camera images may conform to any suitable respective data formats. For example, the acquired medical image data may conform to DICOM format and the camera images may conform to JPEG, MP4, or another suitable format.

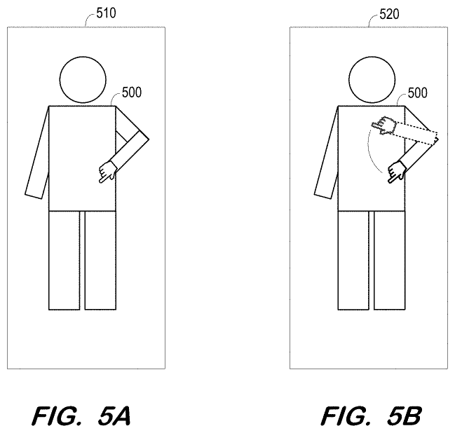

As described above, the camera images may capture patient gestures or markers in the field of view of the camera system. FIGS. 5A and 5B depict camera images 510 and 520 which may be captured in some embodiments of S420. Camera image 510 illustrates patient 500 pointing to a particular portion of the body. This gesture may be intended to illustrate a location of pain, but embodiments are not limited thereto. Camera image 510 may comprise one of a sequence of images acquired at S420 showing patient 500 pointing to the portion of the body.

FIG. 5B depicts several camera images 520 acquired over time. The images 520 depict an arm of patient 500 moving from one location to another. Such a gesture may indicate locations and a direction of pain, but embodiments are not limited thereto. The camera images acquired at S420 may depict information other than patient gestures or movement, such as the placement of markers, for example.

The one or more camera images are converted to a medical imaging format at S430. The medical imaging format to which the camera images are acquired may be the same format to which the medical image data acquired at S410 conforms. With reference to the current example, S430 may comprise conversion of a time-series of JPEG images (i.e., the acquired camera images) to a DICOM series.

The converted one or more camera images are integrated with the acquired internal medical image data at S440. Integration at S440 may facilitate the delivery of the converted one or more camera images and the acquired internal medical image data to a radiologist for viewing. Integration may consist of adding a DICOM series of converted camera images to a file including the one or more DICOM series of acquired internal medical image data.

The integrated data is provided to a medical image data viewing system such as a PACS system at S450. Such provision may occur via a wireless or wired network connection, or delivery of a storage medium storing the data, for example. The integrated data is presented by the medical image data viewing system to a radiologist or other personnel at S460.

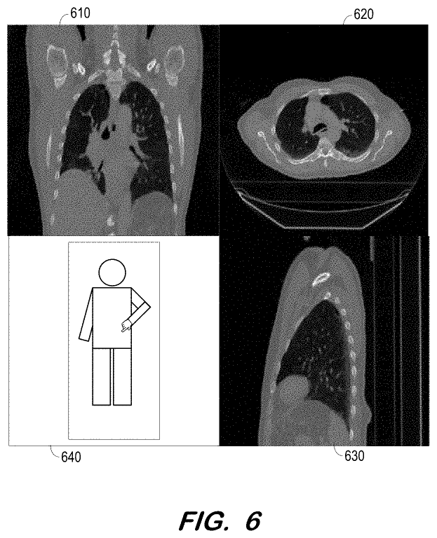

FIG. 6 depicts an example of the integrated data as presented by an image viewing system according to some embodiments. The image viewing system presents three individual DICOM series 610, 620 and 630 of internal medical image data (e.g., axial plane, coronal plane, sagittal plane, external camera video). Also presented is DICOM series 640 consisting of the converted camera image(s). As shown, presentation of the integrated data at S460 facilitates the delivery of information to the radiologist in a manner superior to traditional systems, which may improve subsequent diagnosis and treatment.

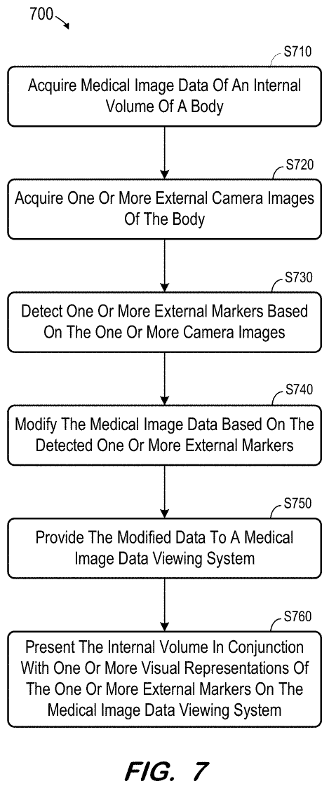

FIG. 7 is a flow diagram of process 700 to identify markers in external camera data and modify medical image data based on the markers. System 250 of FIG. 2B may comprise an implementation of process 700, but embodiments are not limited thereto.

Medical image data of an internal volume of a patient body is acquired at S710 as described above and known in the art. One or more camera images of an external surface of the patient are acquired at S720, as also described above. The acquired medical image data and the acquired camera images may conform to any suitable respective data formats.

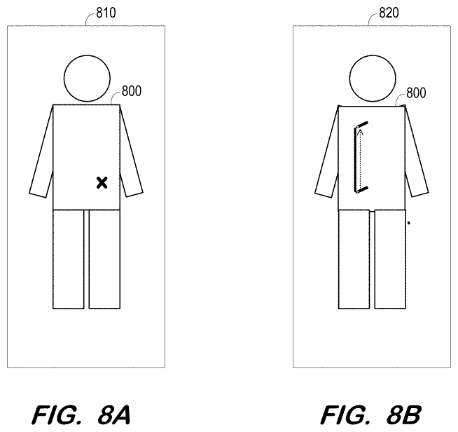

The acquired camera images may capture visual markers placed on the patient or otherwise in the field of view of the camera system. FIGS. 8A and 8B depict camera images 810 and 820 which may be captured in some embodiments of S720. Camera image 810 illustrates an “X” marker placed on patient 500 using any camera-detectable medium (e.g., marker, tape). The marker may be intended to illustrate a location of pain, but embodiments are not limited thereto. Camera image 810 may comprise one of a sequence of images acquired at S720 showing the “X” marker placed on patient 500.

FIG. 8B depicts several camera images 820 acquired over time at S720. The images 820 depict the drawing of a marker on patient 800 from one location to another. This depiction may indicate locations and a direction of pain, but embodiments are not limited thereto. The markers may depict any information to be conveyed to a radiologist in some embodiments.

The one or more external markers are detected at S730 based on the one or more camera images. Detection at S730 may proceed using any suitable image detection techniques that are or become known. Detection at S730 may comprise a determination in three-dimensional space of a location of the detected one or more markers. If the marker changes over time such as in camera images 820 of FIG. 8B, detection at S730 may also comprise a detection of such change.

The medical image data is modified at S740 based on the detected markers at S740. Modification of the medical image data may comprise reconstruction of the medical image data to account for the location of the markers, and/or to include voxels representing the markers in the reconstructed volume. According to some embodiments, the location of the markers is detected at S730 in a three-dimensional frame of reference of the acquiring camera. At S740, the detected location is registered to a location in a frame of reference of an avatar representing the position of the patient, using a known transformation between the two frames of reference as is known in the art. The avatar is then registered to the reconstructed medical image data and this registration is used to transform the location of the markers to the frame of reference of the reconstructed volume.

Since the location of the markers is therefore known in the frame of reference of the reconstructed volume, the reconstructed volume may be modified in any suitable manner to represent the presence of the markers. For example, voxels of the reconstructed volume which are located at the location of the markers and/or adjacent voxels may be modified to represent the markers. Such modification may include coloring the voxels, animating the voxels, adding annotations to the voxels and/or any other suitable modification.

The modified data is provided to a medical image data viewing system such as a PACS system at S750, via a wireless or wired network connection, a tangible storage medium storing the data, or other means. The modified data is presented by the image viewing system at S760. The presented data depicts the internal volume of the body in conjunction with one or more visual representations of the one or more external markers.

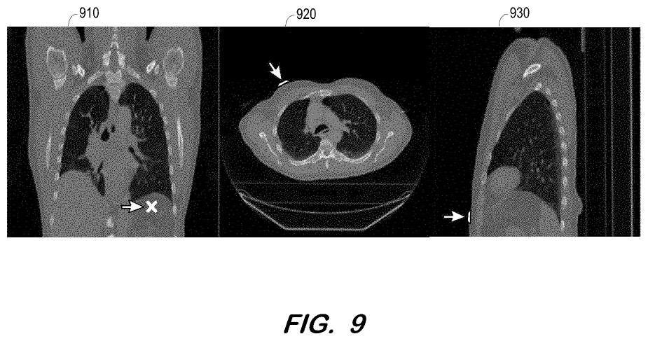

FIG. 9 depicts an example of the modified data as presented by an image viewing system according to some embodiments. The image viewing system presents three individual DICOM series 910, 920 and 930 of internal medical image data. Each of DICOM series 910, 920 and 930 includes white voxels and an arrow which depict the location of a detected external marker. It should be noted that embodiments may employ any suitable visualization of the location of and/or the information to be conveyed by the marker.

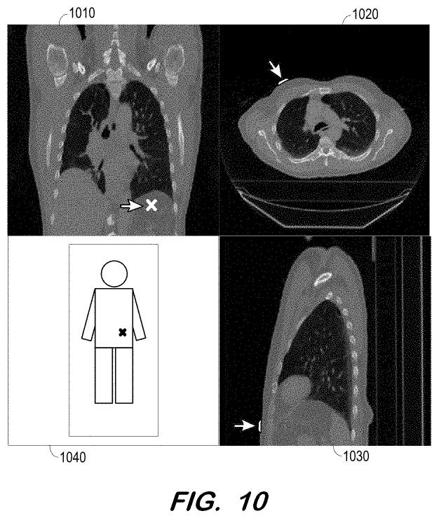

FIG. 10 depicts another example of the modified data as presented by an image viewing system according to some embodiments. Three individual DICOM series 1010, 1020 and 1030 of internal medical image data are presented, including modifications similar to those discussed with respect to DICOM series 910, 920 and 930. Also shown is a third DICOM series 1040 depicting the external camera image acquired at S720. Accordingly, FIG. 10 depicts a system in which the medical image data is modified and presented as described with respect to process 700, and in which the external camera image is also converted to a medical image data format (e.g., DICOM) and integrated with the medical image data as described with respect to process 400.

Presentation of integrated internal medical data and external camera data according to some embodiments facilitates the delivery of information to a radiologist and may improve resulting diagnosis and treatment.

Those in the art will appreciate that various adaptations and modifications of the above-described embodiments can be configured without departing from the scope and spirit of the claims. Therefore, it is to be understood that the claims may be practiced other than as specifically described herein.

What is claimed is:

1. A system comprising:
a medical imaging system to acquire medical image data of an internal volume of a body;
a camera system to acquire camera image data of the body, the camera image data comprising an indication of diagnosis-related information; and
an image processing system to:
receive the medical image data;
receive the camera image data; and
integrate the medical image data and the camera image data to generate integrated medical image data; and
a storage system to store the integrated medical image data.

2. A system according to claim 1, wherein the indication of diagnosis-related information includes a pain location.

3. A system according to claim 1, wherein integration of the medical image data and the camera image data to generate integrated medical image data comprises:
conversion of the camera image data to a format of the medical image data; and
merging of the converted camera image data with the medical image data.

4. A system according to claim 3, wherein the format is Digital Imaging and Communications in Medicine format.

5. A system according to claim 1, wherein integration of the medical image data and the camera image data to generate integrated medical image data comprises:
detection of a marker represented in the camera image data; and
modification of the medical image data to include a representation of the marker.

6. A system according to claim 5, wherein modification of the medical image data to include a representation of the marker comprises:
determination of a first three-dimensional location of the marker in a frame of reference of the camera system;
determination of an avatar of the patient based on the camera image data;
transformation of the first three-dimensional location to a second three-dimensional location in a frame of reference of the avatar; and
transformation of the second three-dimensional location to a third three-dimensional location in a frame of reference of the medical image data.

7. A system according to claim 1, further comprising an image viewing system to present the integrated medical image data, the presented integrated medical image data including the diagnosis-related information.

8. A method, comprising:
acquiring medical image data of an internal volume of a body;
acquiring camera image data of the body, the camera image data comprising an indication of diagnosis-related information.
integrating the medical image data and the camera image data to generate integrated medical image data; and
storing the integrated medical image data.

9. A method according to claim 8, wherein the camera image data includes a pain location.

10. A method according to claim 8, wherein integrating the medical image data and the camera image data to generate integrated medical image data comprises:
converting the camera image data to a format of the medical image data; and
merging the converted camera image data with the medical image data.

11. A method according to claim 10, wherein the format is Digital Imaging and Communications in Medicine format.

12. A method according to claim 8, wherein integrating the medical image data and the camera image data to generate integrated medical image data comprises:
detecting a marker represented in the camera image data; and
modifying the medical image data to include a representation of the marker.

13. A method according to claim 12, wherein modifying the medical image data to include a representation of the marker comprises:
determining a first three-dimensional location of the marker in a frame of reference of the camera system;
determining an avatar of the patient based on the camera image data;
transforming the first three-dimensional location to a second three-dimensional location in a frame of reference of the avatar; and
transforming the second three-dimensional location to a third three-dimensional location in a frame of reference of the medical image data.

14. A method according to claim 8, further comprising displaying the integrated medical image data, the displayed integrated medical image data including the diagnosis-related information.

15. A non-transitory computer-readable medium storing processor-executable process steps, the process steps executable by a processor to cause a system to:
acquire medical image data of an interval volume of a body;
acquire camera image data of the body, the camera image data comprising an indication of diagnosis-related information.
integrate the medical image data and the camera image data to generate integrated medical image data; and
display the integrated medical image data.

16. A medium according to claim 15, wherein the camera image data includes a pain location.

17. A medium according to claim 15, wherein integration of the medical image data and the camera image data to generate integrated medical image data comprises:
conversion of the camera image data to a format of the medical image data; and
merging of the converted camera image data with the medical image data.

18. A medium according to claim 17, wherein the format is Digital Imaging and Communications in Medicine format.

19. A medium according to claim 15, wherein integration of the medical image data and the camera image data to generate integrated medical image data comprises:
detection of a marker represented in the camera image data; and
modifying of the medical image data to include a representation of the marker.

20. A medium according to claim 19, wherein modification of the medical image data to include a representation of the marker comprises:
determination of a first three-dimensional location of the marker in a frame of reference of the camera system;
determination of an avatar of the patient based on the camera image data;
transformation of the first three-dimensional location to a second three-dimensional location in a frame of reference of the avatar; and
transformation of the second three-dimensional location to a third three-dimensional location in a frame of reference of the medical image data.
